(19) United States
(12) Patent Application Publication
(10) Pub. No.: US 2006/0281976 A1
(43) Pub. Date: Dec. 14, 2006

(54) Method for Exercise Tolerance Measurement

(76) Inventors: Jer-Nan Juang, Yun-Lin Hsien (TW); Han-Pin Kuo, Taipei City (TW); Wen-Te Liu, Tao-Yuan Hsien (TW); Shu-Hui Hung, Tai-Chung Hsien (TW); Kang-Yang Liang, Taipei City (TW)

Correspondence Address: Alan D. Kamrath;NIKOLAI & MERSEREAU, P.A., Suite 820 International Centre, 900 Second Avenue South, Minneapolis, MN, 55402, US

(21) Appl. No.: 11/161,751

(22) Filed: Aug. 16, 2005

(30) Foreign Application Priority Data
Jun. 7, 2005 (TW) 094118791

(51) Int. Cl. A61B 5/00; A61B 5/08

(52) U.S. Cl. 600/300; 600/559; 600/529; 128/903

(57) ABSTRACT

An exercise tolerance measurement method for sufferers of chronic obstructive airway disease is established with the aid of a wireless transmission system and the Internet. The exercise tolerance measurement method provides sufferers of chronic obstructive airway disease with personal homely rehabilitation exercises and real-time on-line diagnosis policy according to the conditions of the sufferers. More importantly, the conditions of the sufferers can be monitored immediately via the Internet by the present measurement method. In the case of any emergency, the claimed method notifies the sufferers or the physician to supply instant medical treatment to the sufferers.

### BACKGROUND OF THE INVENTION

1. Field of the Invention

The present invention relates to a method for exercise tolerance measurement, more particularly, a testing method for exercise tolerance measurement that a patient is capable of performing at home.

2. Description of the Prior Art

Chronic obstructive airway disease, including chronic obstructive asthma and chronic obstructive pulmonary disease, are the most common chronic respiratory diseases. In the modern society, with the increasing age of the population, the growing industrialization, and the increasing air pollution, the population infected with chronic obstructive airway disease will increase. World Heath Organization (WHO) estimated that by the year 2020, the world population infected with chronic obstructive airway disease will grow to more than ten percent, and death rates and medical expenses will be ranked fourth among all diseases. Therefore, the chronic obstructive airway disease is one of the serious diseases faced by the medical professions today.

The main cause for chronic obstructive airway disease is long and constant exposure to breathing either cigarette smoke or polluted air. The resulting chronic respiratory inflammation caused by this exposure will lead to changes in the structure of the respiratory system. This is especially evident when the bronchus is blocked or the pulmonary alveolus structure is damaged. In either of these cases, the vapor in the lungs is unable to evacuate and as a result, emphysema is formed. A patient does not directly sense anything unusual during the initial stage of damage; however, a reduction in the level of the patient's exercise tolerance can definitely be felt. As the respiratory tract becomes infected then symptoms of flam and breathing difficulty will begin to appear. Progressively the patient's exercise tolerance level and breathing difficulty will worsen and finally the patient must only rely on oxygen. At this point, the patient cannot perform day-to-day activities and is at risk of the collapse of the cardiopulmonary system.

Exercise tolerance will usually drop before any acute symptoms break out. Therefore, regularly monitoring exercise tolerance of the patient is an effective method for detecting the acute symptoms of chronic obstructive airway disease. However, most exercise tolerance test can only be performed at a hospital. It is difficult for patients that stay at home to benefit from an exercise tolerance test. Please refer to FIG. 1. FIG. 1 illustrates a diagram of an exercise tolerance test performed according to a conventional detecting method. In the conventional exercise tolerance test, a patient or a person being tested 10 is performing the exercise tolerance test in a hospital and usually with the surveillance of medical staff. As shown in FIG. 1, when the patient 10 is performing the test, the patient 10 walks around two taper cones 12 with a diameter of nine meters, and when the patient walks from one taper cone 12 to another taper cone 12, the walking distance is approximately ten meters. There will be a ring tone that sounds during the walking process. The patient must walk around to the other taper cone 12 before the next ring tone sounds. The time interval between the rings may be ten seconds to avoid the starting ring from being misinterpreted by the patient thereby causing the test to fail resulting in medical staff being required to intercede to remind the patient 10 about the next ring so that the test can be completed. During the test, the time interval between rings is reduced causing the patient to walk at a quicker pace to reach the next taper cone 12 before the next ring. Once the test is started, the patient 10 must continue walking until the patient 10 cannot maintain the current pace with normal breathing circumstances. Furthermore, during the next ring, if the patient's 10 distance is 0.5 meters or more from reaching the next taper cone 12, then the test is stopped. Lastly, the exercise tolerance of the patient 10 can be determined according to the walking speed when the test is stopped.

Please refer to FIG. 2. FIG. 2 illustrates a conventional exercise tolerance test performed according to a test list 20. The test list 20 defines different test levels, walking speeds, time intervals between rings and so forth. Level 1 begins at a walking speed of 0.5 meters per second with the levels continue until level 12. The walking speed increases by 0.27 meters per second every time the level is increased. The trip interval column in the test list 20 represents a maximum time (in seconds) allotted for the patient to walk around the two taper cones; from the column we can know that as the test level increases less time (in seconds) is provided for walking 10 meters, hence the patient's walking speed increases.

In the above-mentioned, there remain limitations when realizing the method; for example, the patient is usually required to visit a hospital to perform measurement. Often the patient may feel that this activity will be dull therefore, the patient will be uninterested in the test. Given the conventional method, it is difficult to understand and access the situation with the patient located at home. As a result, it is difficult to ask the patient to visit the hospital to perform the test. Additionally, when doctors diagnose the patient according to the test results, the reference value of the test results may not be as accurate as they seem.

### SUMMARY OF THE INVENTION

Therefore, the objective of the claimed invention is to provide a method for exercise tolerance measurement to overcome the defect of the problem in the above-mentioned prior art.

The claimed invention discloses a method for exercise tolerance measurement, the method comprising generating corresponding test parameters according to a condition generated after an exercise status test is performed on a patient, generating a test strategy according to the test parameters generated, playing tunes according to the test strategy generated, and analyzing the exercise status of the patient according to a condition of playing the tunes.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 illustrates a diagram of an exercise tolerance test performed according a conventional detecting method.

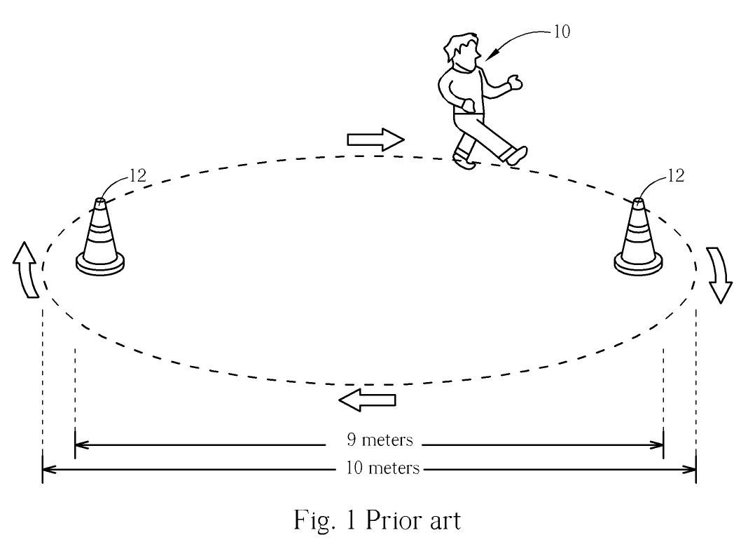

FIG. 2 illustrates a conventional exercise tolerance test performed according to a test list.

FIG. 3 illustrates a flowchart of a method of exercise tolerance being tested on a patient.

FIG. 4 illustrates a diagram of a treatment system according to the method of the present invention.

FIG. 5 illustrates a diagram of a patient performing the method of the present invention.

FIG. 6 illustrates a test list according to an embodiment of the present invention.

FIG. 7 illustrates a comparison table utilized in FIG. 4.

### DETAILED DESCRIPTION

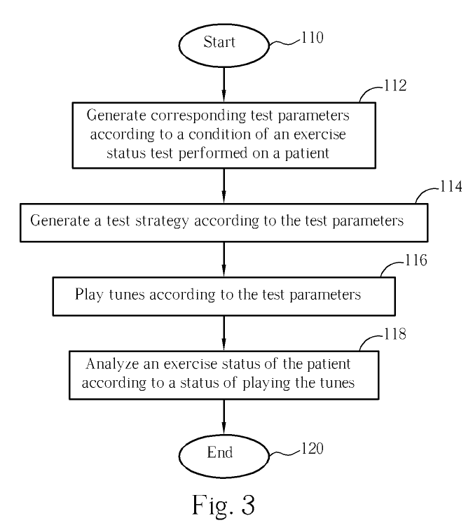

Please refer to FIG. 3. FIG. 3 illustrates a flowchart of a method of exercise tolerance being tested on a patient. In order to achieve the objective of the patient performing the exercise tolerance test at home and without the supervision of medical staff, an exercise status test must be performed on the patient first. The aim of this exercise status test is to understand each individual condition of the patient, for example: the length of each step. The present invention then generates corresponding test parameters (step 112) according to the condition of the exercise status test performed on the patient. The exercise status test includes, for example: recording different walking speeds of the patient walking the ten meters, recording the number of steps taken to walk the ten meters at different speeds, recording the results of different speeds and corresponding steps. The recorded results will be utilized as the patient's test parameters. In this embodiment, after the test parameters have been set, the method of the present invention generates a test strategy (step 114) according to the test parameters, hence the test parameters define a specific sequence of certain tunes to be played during the test. The patient can play the tunes (step 114) using a handheld player (such as a mobile phone) according to the tunes defined in the test strategy (step 116). When the tunes are played, the patient will move according to the tempo of the tunes. The effect being that the tempo of the tunes is utilized to affect the walking speed of the patient. In this embodiment the tempo of the tunes will increase according to the progress of the test thereby guiding the patient to quicken the walking pace. At the time when the patient cannot maintain the current pace under normal breathing circumstances, the patient can stop the exercise tolerance test. Lastly, the patient's exercise status during the test can be analyzed (step 118) according to the tempo of the tunes played to determine the exercise tolerance of the patient.

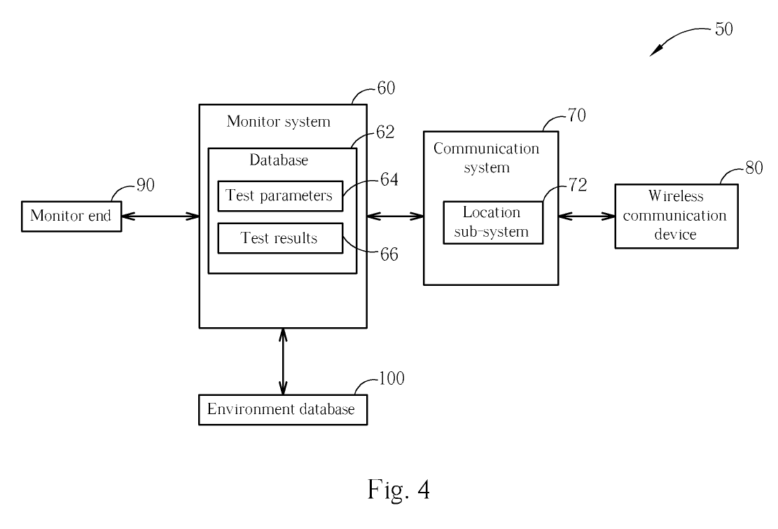

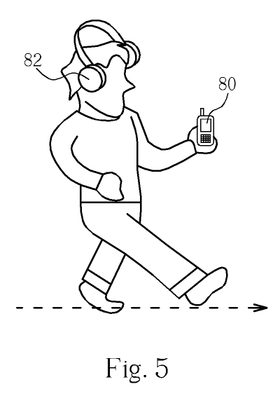

The method of the present invention can be further realized by utilizing wireless communication technology and the Internet. Please refer to FIG. 4. FIG. 4 illustrates a diagram of a treatment system 50 according to the method of the present invention. The treatment system 50 comprises a monitor system 60, a communication system 70, a wireless communication device 80, a monitor end 90 and an environment database 100. The monitor system 60, the communication system 70, and the wireless communication device 80 perform data transmission in a wireless manner. The monitor system 60, the monitor end 90, and the environment database are connected through the Internet. The monitor system 60 is primarily responsible for integration and connectivity among each system and each device in the treatment system 50. The function of the communication system 70 is to establish a communication mechanism between the wireless communication device 80 utilized by the monitor system 60 and the patient. After the exercise status test is completed by the patient, the test parameters 64 generated will be stored into a database 62 of the monitor system 60. When the patient utilizes the wireless communication device 80 to send a test request to the monitor system 60 via the communication system 70, the monitor system 60 will differentiate the patient's identity according to the test request received, and the monitor system 60 will search for the test parameters corresponding to the patient's identification database 62 and draft a test strategy according to the test parameters 64 obtained. The drafted test strategy will then be transmitted to the wireless communication device 80 in a wireless manner via the communication system 70. When the communication device 80 receives the test strategy from the monitor system 60 via the communication system 70, the corresponding tunes will be uploaded from a memory to be played according to the test strategy to perform the exercise tolerance test. Please refer to FIG. 5. FIG. 5 illustrates a diagram of a patient performing the method of the present invention. The patient listens to the tune indicated by the monitor system through earphones 82 connected to the wireless communication device 80. While listening to the tune, the patient walks at a pace corresponding to the tempo of the tune. It is evident that when the patient is performing the exercise tolerance test, the location of the test is not limited and supervision is not required. To further explain the present invention, the patient is not limited to listening to the tunes via earphones 82. Alternatively, the patient can listen to the tunes via internal speakers in the wireless communication device 80. Please refer to FIG. 4 again. In the test process, the tempo of the tunes played by the wireless communication device 80 will gradually become faster and similarly, when the patient cannot keep up with the speed under normal breathing circumstances, the patient can stop the exercise tolerance test. When the patient stops the test, a stop test signal is required to be sent to the monitor system 60 by the wireless communication device 80, the monitor system 60 then can analyze the patient's status during the test according to the test request and the stop test signal sent out by the wireless communication device 80. Data of test results 66 obtained which is analyzed by the monitor system 60 comprises the length of time taken for the patient to complete the test and the speed of the patient at the end of the test; the monitor system 60 can predict the exercise tolerance of the patient according to the test results 66. Furthermore, if the monitor system 60 analyzes the patient's status and determines the status to be abnormal according to the test results 66 of the patient, the communication system 70 will contact the patient or the patient's doctor directly to provide medical help and attention.

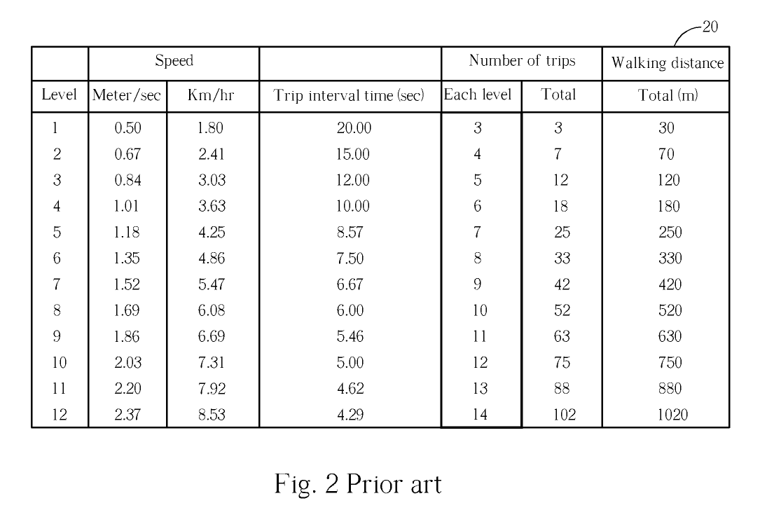

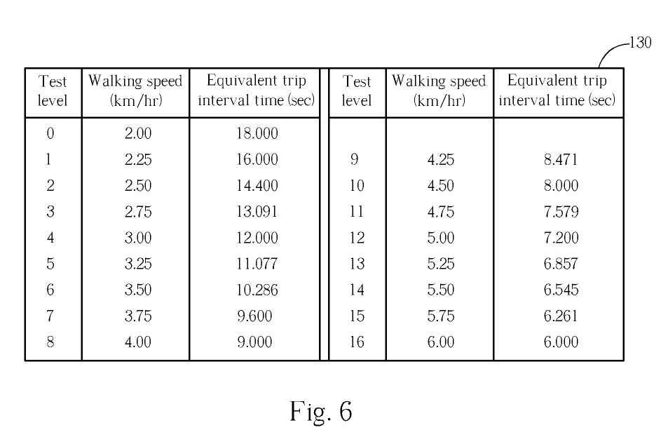

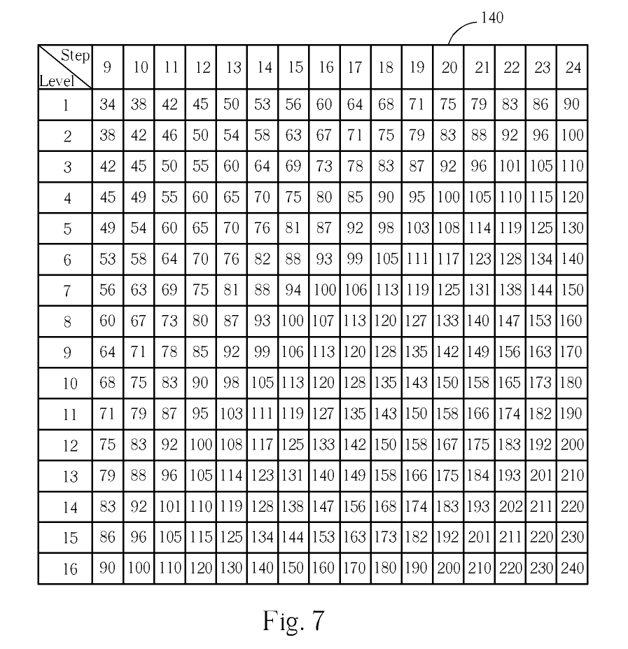

Please refer to FIG. 6 and FIG. 7. FIG. 6 illustrates a test list 130 according to an embodiment of the present invention. FIG. 7 illustrates a comparison table 140 utilized in FIG. 4. The test list 130 of FIG. 6 defines the walking speed as it corresponds to each test level; wherein the equivalent trip interval time is defined as the time required to complete ten meters with respect to the walking speed. The corresponding walking speeds and levels can be found in the test list 20 in FIG. 2 utilizing the equivalent trip interval time defined in the test list 130. The comparison table 140 shown in FIG. 7 can help to record the tempo of each tune (i.e., the beats per minute of the tune) played during each test level according to the patient's individual condition. The step length (i.e., gait) of each patient varies. To prevent different step lengths from affecting the accuracy of the walking speed of the patient as calculated by the monitor system 60 the variable step length of the patient will be considered. Row of steps in the comparison table 140 hence corresponds with the test parameters generated in step 112 of FIG. 3. This is the number of steps the patient will take to walk 10 meters. When the patient is performing the exercise tolerance test, the monitor system 60 selects tunes suitable for the patient to hear during the test according to the patient's test parameters (e.g., information regarding the patient's step length) and the corresponding tune tempo recorded in the comparison table 140. For example, the method of the present invention can be set to one minute per level, and the monitor system 60 selects tunes that are suitable for the speed required for each level and that are to be played for one minute. After a tune has completed then that particular level is completed. The wireless communication device 80 continues to play a next tune corresponding to speed of the next level to guide the patient to increase speed.

The method for exercise tolerance of the present invention, other than the functions of the above-mentioned, consists of other functions. Please refer to FIG. 4 again. As shown in FIG. 4, the communication system 70 of the treatment system 50 further comprises a location sub-system 72 for providing location of the wireless communication device 80 utilized by the patient. The monitor system 60 can read the related environment information (e.g., air quality, temperature, weather conditions) of the location of the patient from the environment database 100 according to the location information of the wireless communication device 80 provided by the location sub-system 72. The environment information then is transmitted to the wireless communication device 80 via the communication system 70 for the patient's reference. Alternatively, the monitor system 60 is also capable of providing related treatment recommendations, for example related medication and exercise for the patient according to the environment information read and the test results 66 of the patient. Additionally, the patient's doctor can understand the patient's current condition by reading the test results 66 from the monitor system 60 via the monitor end 90 to provide the best treatment recommendation to the patient.

In comparison to the prior art, the method for exercise tolerance measurement provided by the present invention generates a corresponding test parameters according to a condition generated after an exercise status test is performed on a patient to suit an individual condition of the patient to proceed with a test. The method of the present invention then utilizes the test parameters to select suitable tunes for the test so that the patient can walk at a pace to match the tempo of the tunes that are played during the test, as the tunes will replace the ring tone previously used the patient will not get frustrated as easily during the test period which encourages the patient to proceed with the test. Furthermore, the patient can listen to the tunes via a wireless communication device and the patient is not required to perform the exercise tolerance measurement at specialized location such as the hospital; the patient can perform the test anywhere without location restrictions.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A method for exercise tolerance measurement, the method comprising the following steps:
(a) generating corresponding test parameters according to a condition generated after an exercise status test is performed on a patient;
(b) generating a test strategy according to the test parameters generated in step (a);
(c) playing tunes according to the test strategy generated in step (b); and
(d) analyzing an exercise status of the patient according to a condition of playing the tunes in step (c).

2. The method of claim 1 wherein a monitor system in step (b) generates the test strategy according to the test parameters generated according to step (a).

3. The method of claim 2 further comprising requesting the monitor system to execute the test strategy utilizing a wireless communication device in a wireless manner.

4. The method of claim 3 wherein the monitor system in step (c) controls the wireless communication device in a wireless manner to play the tunes corresponding to the test strategy.

5. The method of claim 4 further comprising reporting the condition of playing the tunes in step (c) via the wireless communication device to the monitor system.

6. The method of claim 5 wherein the monitor system in step (d) analyzes the exercise status of the patient according to the condition of playing the tunes in step (c).

7. The method of claim 6 wherein the monitor system in step (d) analyzes the exercise status of the patient according to signals transmitted by the wireless communication device and the condition of playing the tunes in step (c).

8. The method of claim 3 further comprising transmitting a measurement notice, at a preset time, generated by the monitor system to the wireless communication device.

9. The method of claim 3 wherein the monitor system in step (b) generates the test strategy according to signals transmitted by the wireless communication device and the test parameters generated in step (a).

10. The method of claim 3 further comprising the following step:
(e) generating a treatment recommendation according to a result analyzed in step (d).

11. The method of claim 10 wherein the monitor system in step (e) generates the treatment recommendation according to signals transmitted by the wireless communication device and the result analyzed in step (d).

12. The method of claim 11 further comprising transmitting the treatment recommendation generated by the monitor system in step (e) to the wireless communication device in a wireless manner.

13. The method of claim 1 wherein step (c) plays tunes from a slow to fast beat according to the test strategy generated in step (b).
